(19) United States
(12) Patent Application Publication
(10) Pub. No.: US 2025/0352273 A1
(43) Pub. Date: Nov. 20, 2025

(54) IMAGE PROCESSING APPARATUS, MEDICAL SYSTEM, OPERATION METHOD OF IMAGE PROCESSING APPARATUS, AND LEARNING APPARATUS

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Toshiki MIZUMAKI, Tokyo (JP)

(73) Assignee: OLYMPUS MEDICAL SYSTEMS CORP., Tokyo (JP)

(21) Appl. No.: 19/289,549

(22) Filed: Aug. 4, 2025

Related U.S. Application Data
(63) Continuation of application No. PCT/JP2023/004396, filed on Feb. 9, 2023.

(51) Int. Cl. A61B 34/10; A61B 1/00; A61B 1/04; A61B 1/06

(52) U.S. Cl. CPC A61B 34/10; A61B 1/000096; A61B 1/0005; A61B 1/043; A61B 1/0638; A61B 2034/104; A61B 2034/105

(57) ABSTRACT

An image processing apparatus includes: a processor including hardware. The processor is configured to acquire a white light image and a fluorescence image for a living tissue, acquire output information of an energy device, set a first threshold in accordance with the output information, estimate an auxiliary line for incising the living tissue based on first positional information corresponding to a pixel in the fluorescence image having a luminance value equal to or larger than the first threshold, and generate information in which the auxiliary line is superimposed on the white light image.

### CROSS REFERENCES TO RELATED APPLICATIONS

This application is a continuation of International Application No. PCT/JP2023/004396, filed on Feb. 9, 2023, the entire contents of which are incorporated herein by reference.

### BACKGROUND

### 1. Technical Field

The present disclosure relates to an image processing apparatus, a medical system, an operation method of the image processing apparatus, and a learning apparatus.

### 2. Related Art

In the related art, in the medical field, minimally invasive operation using an endoscope, a laparoscope, or the like is widely performed. For example, as the minimally invasive operation using the endoscope, the laparoscope, or the like, an Endoscopic Submucosal Dissection (ESD) is widely performed.

In the ESD, a living tissue is cauterized by an energy device or the like, and therefore, a plurality of dotted markings are formed so as to enclose a lesion site that is an excision target. The living tissue that is thermally denatured by the markings contains advanced glycation end products (AGEs) and emits fluorescence when being applied with excitation light, and therefore, it is possible to confirm positions of the markings by a fluorescence image (for example, see International Publication No. 2020/054723). Further, an operator determines an incision line by using the positions of the markings as marks and makes an incision along the incision line to excise the lesion site.

### SUMMARY

In some embodiments, an image processing apparatus includes: a processor including hardware, the processor being configured to acquire a white light image and a fluorescence image for a living tissue, acquire output information of an energy device, set a first threshold in accordance with the output information, estimate an auxiliary line for incising the living tissue based on first positional information corresponding to a pixel in the fluorescence image having a luminance value equal to or larger than the first threshold, and generate information in which the auxiliary line is superimposed on the white light image.

In some embodiments, a medical system includes: a light source configured to apply while light and excitation light to a living tissue; an endoscope that includes an image sensor configured to output a first imaging signal that is obtained by imaging return light of the white light and a second imaging signal that is obtained by imaging fluorescence caused by the excitation light; and the image processing apparatus. The processor of the image processing apparatus is further configured to generate a white light image from the first imaging signal and a fluorescence image from the second imaging signal.

In some embodiments, provided is an operation method of an image processing apparatus. The method includes: acquiring a white light image and a fluorescence image for a living tissue, acquiring output information of an energy device, setting a first threshold in accordance with the output information, estimating an auxiliary line for incising the living tissue based on positional information corresponding to a pixel in the fluorescence image having a luminance value equal to or larger than the first threshold, and generating information in which the auxiliary line is superimposed on the white light image.

In some embodiments, a learning apparatus includes: a learning processor configured to generate a trained model by performing machine learning by using teacher data in which a white light image and a fluorescence image for a living tissue are adopted as input data and information in which an auxiliary line for incising the living tissue is superimposed on the white light image is adopted as output data.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an overall configuration of an endoscope system according to one embodiment;

FIG. 2 is a block diagram illustrating a functional configuration of a main part of the endoscope system according to one embodiment;

FIG. 3 is a flowchart illustrating an outline of a process that is performed by a control apparatus;

FIG. 4 is a diagram illustrating an example of a fluorescence image;

FIG. 5 is a diagram illustrating an example of an incision line;

FIG. 6 is a diagram illustrating a state in which the incision line is superimposed on a white light image;

FIG. 7 is a diagram illustrating an example of an incised line;

FIG. 8 is a diagram illustrating a state in which a part of the incision line is deleted;

FIG. 9 is a diagram illustrating an example of the incised line; and

FIG. 10 is a diagram illustrating a state in which a part of the incision line is deleted.

### DETAILED DESCRIPTION

As modes (hereinafter, referred to as “embodiments”) for carrying out the present disclosure, an endoscope system that includes an endoscope with a flexible insertion portion will be described below, but embodiments are not limited this example, and may be applied to, for example, a rigid scope, a surgical robot, or the like. Further, the present disclosure is not limited by the embodiments below. Furthermore, in description of the drawings, the same components are denoted by the same reference symbols. Moreover, it is necessary to note that the drawings are schematic, and relations between thicknesses and widths of the components, ratios among the components, and the like may be different from actual ones. Furthermore, the drawings may include portions that have different dimensional relations or ratios.

### Configuration of Endoscope System

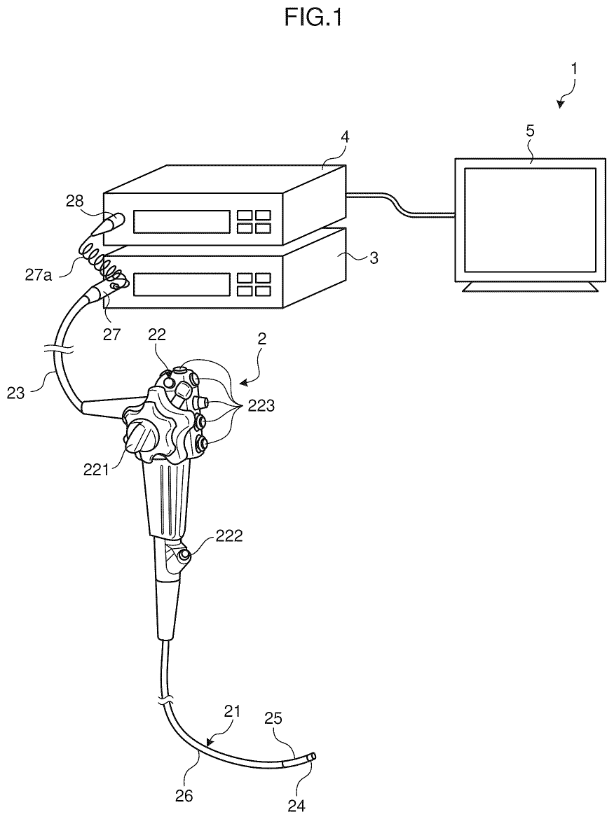

FIG. 1 is a diagram schematically illustrating an overall configuration of an endoscope system according to one embodiment. An endoscope system 1 illustrated in FIG. 1 captures an image inside a body of a subject, such as a patient, by inserting an insertion portion of an endoscope into a body cavity or a lumen of the subject, and displays a display image that is based on a captured imaging signal on a display apparatus. The endoscope system 1 includes an endoscope 2, a light source apparatus 3, a control apparatus 4 as an image processing apparatus, and a display apparatus 5.

### Configuration of Endoscope

A configuration of the endoscope 2 will be described below.

The endoscope 2 generates an imaging signal (RAW data) by performing imaging inside a body of a subject, and outputs the generated imaging signal to the control apparatus 4. Specifically, the endoscope 2 generates a first imaging signal by applying white light and imaging a return light, and a second imaging signal by applying excitation light and imaging fluorescence. The endoscope 2 includes an insertion portion 21, an operation unit 22, and a universal cord 23.

The insertion portion 21 is inserted into the subject. The insertion portion 21 has a flexible thin and elongated shape. The insertion portion 21 includes a distal end portion 24 that includes a built-in image sensor (to be described later), a bending portion 25 that includes a plurality of bending pieces and that is bendable, and a flexible tube portion 26 that is connected to a proximal end of the bending portion 25 and that has a flexible elongated shape.

The distal end portion 24 is configured with a fiberglass or the like. The distal end portion 24 serves as a light guide for illumination light that is supplied from the control apparatus 4 via the universal cord 23 and the operation unit 22, generates an imaging signal by imaging return light of the illumination light, and outputs the imaging signal to the control apparatus 4.

The operation unit 22 includes a bending knob 221 that causes the bending portion 25 to bend in a vertical direction and in a horizontal direction, a treatment tool insertion portion 222 for inserting a body treatment tool, and a plurality of switches 223 that are operation input units for inputting an operation instruction signal for the control apparatus 4 or a peripheral device, such as an air supply means, a water supply means, or a gas supply means, a pre-freeze signal for instructing the endoscope system 1 to capture a still image, or a switching signal for switching an observation mode of the endoscope system 1. The treatment tool that is inserted from the treatment tool insertion portion 222 gets out of an opening portion (not illustrated) through a treatment tool channel (not illustrated) of the distal end portion 24.

The universal cord 23 includes at least a light guide and an assembly cable in which one or a plurality of cables are collected. The assembly cable is a signal line for transmitting and receiving a signal between the endoscope 2 and the control apparatus 4, and includes a signal line for transmitting and receiving an imaging signal (RAW data), a signal line for transmitting and receiving a driving timing signal (a synchronous signal or a clock signal) for driving an image sensor (to be described later). The universal cord 23 includes a connector 27 that is attachable to and detachable from the control apparatus 4, an extended coil cable 27a that has a coil shape and that extends from the connector 27, and a connector 28 that is arranged at an extended end of the coil cable 27a and that is attachable to and detachable from the control apparatus 4.

### Configuration of Light Source Apparatus

A configuration of the light source apparatus will be described below.

The light source apparatus 3 applies, as illumination light, white light and excitation light to a living tissue. The light source apparatus 3 is connected to one end of the light guide of the endoscope 2 and supplies the illumination light that is applied to the inside of the subject to the one end of the light guide, under the control of the control apparatus 4. The light source apparatus 3 is implemented by using at least one of light sources such as a Light Emitting Diode (LED) light source, a xenon lam, and a semiconductor laser device including a Laser Diode (LD), a processor that is a processing apparatus that includes hardware, such as a Field Programmable Gate Array (FPGA) or a Central Processing Unit (CPU), and a memory that is a temporary storage area that is used by the processor. Meanwhile, the light source apparatus 3 and the control apparatus 4 may be configured to perform communication individually as illustrated in FIG. 1, or may be configured in an integrated manner.

### Configuration of Control Apparatus

A configuration of the control apparatus 4 will be described below.

The control apparatus 4 controls each of the units of the endoscope system 1. The control apparatus 4 supplies illumination light that is applied to the subject by the endoscope 2. Further, the control apparatus 4 performs various kinds of image processing on an imaging signal that is input from the endoscope 2, and outputs the imaging signal to the display apparatus 5.

### Configuration of Display Apparatus

A configuration of the display apparatus 5 will be described below.

The display apparatus 5 displays a display image based on a video signal that is input from the control apparatus 4, under the control of the control apparatus 4. The display apparatus 5 is implemented by a display panel made of organic Electro Luminescence (EL), liquid crystal, or the like.

### Functional Configuration of Main Part of Endoscope System

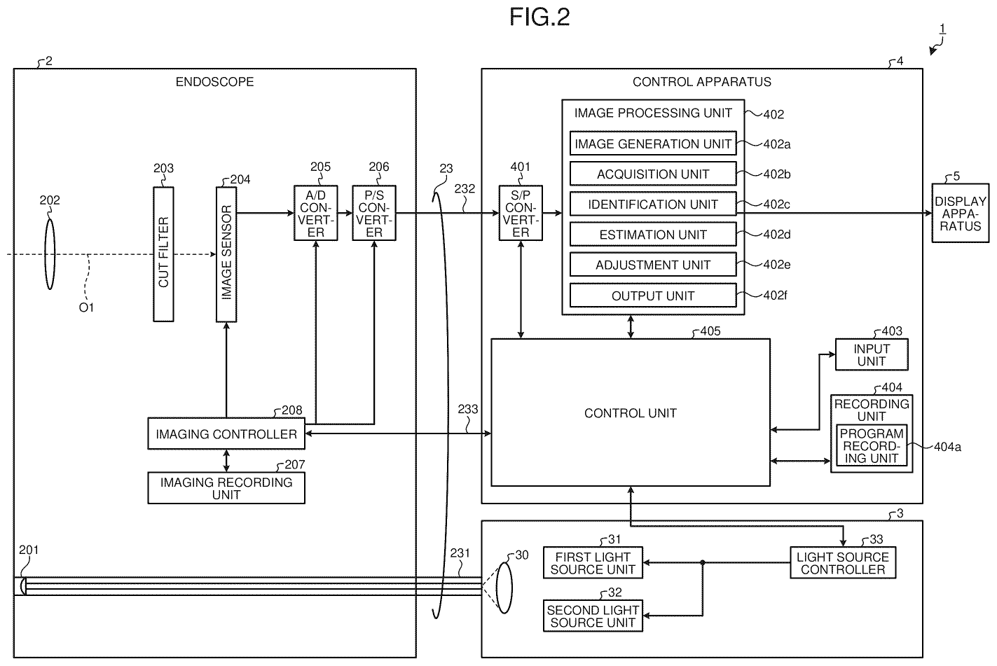

A functional configuration of a main part of the endoscope system 1 as described above will be described below. FIG. 2 is a block diagram illustrating a functional configuration of the main part of the endoscope system 1.

### Configuration of Endoscope

A configuration of the endoscope 2 will be described below.

The endoscope 2 includes an illumination optical system 201, an imaging optical system 202, a cut filter 203, an image sensor 204, an analog-to-digital (A/D) converter 205, a parallel-to-serial (P/S) converter 206, an imaging recording unit 207, and an imaging controller 208. Meanwhile, each of the illumination optical system 201, the imaging optical system 202, the cut filter 203, the image sensor 204, the A/D converter 205, the P/S converter 206, the imaging recording unit 207, and the imaging controller 208 is arranged inside the distal end portion 24.

The illumination optical system 201 applies illumination light that is supplied from a light guide 231 that is formed of an optical fiber or the like to a subject (living tissue). The illumination optical system 201 is implemented by one or a plurality of lenses or the like.

The imaging optical system 202 condenses reflected light that is reflected from the subject, return light that comes from the subject, fluorescence that is emitted by the subject, or the like and forms an object image (light beams) on a light receiving surface of the image sensor 204. The imaging optical system 202 is implemented by one or a plurality of lenses or the like.

The cut filter 203 is arranged on an optical axis O1 of the imaging optical system 202 and the image sensor 204. The cut filter 203 blocks light in a wavelength band of reflected light or a return light from the subject with respect to excitation light that is supplied from the light source apparatus 3, and transmits light in a wavelength band on the long-wavelength side as compared to the wavelength band of the excitation light.

The image sensor 204 receives the object image (light beams) that is formed by the imaging optical system 202 and that is transmitted through the cut filter 203, performs photoelectric conversion to generate an imaging signal (RAW data), and outputs the imaging signal to the A/D converter 205, under the control of the imaging controller 208. The image sensor 204 is implemented by an image sensor, such as a Charge Coupled Device (CCD) or a Complementary Metal Oxide Semiconductor (CMOS), in which any of color filters of Bayer arrangement (RGGB) is arranged on each of pixels that are arranged in a two-dimensional matrix manner.

The A/D converter 205 performs A/D conversion processing on an analog imaging signal that is input from the image sensor 204, and outputs the imaging signal to the P/S converter 206, under the control of the imaging controller 208. The A/D converter 205 is implemented by an A/D conversion circuit or the like.

The P/S converter 206 performs parallel-to-serial conversion on a digital imaging signal that is input from the A/D converter 205, and outputs the imaging signal that is subjected to the parallel-to-serial conversion to the control apparatus 4 via a first transmission cable 232, under the control of the imaging controller 208. The P/S converter 206 is implanted by a P/S conversion circuit or the like. Meanwhile, in the first embodiment, it may be possible to arrange, instead of the P/S converter 206, an electrical-to-optical (E/O) converter that converts an imaging signal to an optical signal and output the imaging signal by the optical signal to the control apparatus 4, or it may be possible to transmit the imaging signal to the control apparatus 4 by radio communication, such as Wireless Fidelity (Wi-Fi) (registered trademark), for example.

The imaging recording unit 207 records therein various kinds of information on the endoscope 2 (for example, pixel information on the image sensor 204 and characteristics of the cut filter 203). Further, the imaging recording unit 207 records therein various kinds of setting data and control parameters that are transmitted from the control apparatus 4 via a second transmission cable 233. The imaging recording unit 207 is configured with a non-volatile memory or a volatile memory.

The imaging controller 208 controls operation of each of the image sensor 204, the A/D converter 205, and the P/S converter 206 based on setting data that is received from the control apparatus 4 via the second transmission cable 233. The imaging controller 208 is implemented by a Timing Generator (TG), a processor that is a processing apparatus that includes hardware, such as a CPU, and a memory that is a temporary storage area that is used by the processor.

### Configuration of Light Source Apparatus

A configuration of the light source apparatus 3 will be described below.

The light source apparatus 3 includes a condenser lens 30, a first light source unit 31, a second light source unit 32, and a light source controller 33.

The condenser lens 30 condenses light that is emitted by each of the first light source unit 31 and the second light source unit 32, and emits the condensed light to the light guide 231. The condenser lens 30 is configured with one or a plurality of lenses.

The first light source unit 31 emits white light (normal light) that is visible light and supplies the white light as illumination light to the light guide 231, under the control of the light source controller 33. The first light source unit 31 is configured with a collimator lens, a white LED lamp, a driving driver, and the like. Meanwhile, the first light source unit 31 may supply white light that is visible light by causing a red LED lamp, a green LED lamp, and a blue LED lamp to simultaneously emit light. The first light source unit 31 may of course be configured with a halogen lamp, a xenon lamp, or the like. Further, the first light source unit 31 may apply reference light with a wavelength that is included in a wavelength band of the white light and that does not include a wavelength band of the fluorescence. By performing imaging by applying the reference light at the same time as the excitation light that is applied by the second light source unit 32, it is possible to perform positional alignment between a white light image and a reference light image.

The second light source unit 32 emits excitation light in a predetermined wavelength band and supplies the excitation light as illumination light to the light guide 231, under the control of the light source controller 33. Here, the excitation light has a wavelength that causes a substance, such as Advanced Glycation End Products (AGEs), that is contained in a thermally denatured region, and a wavelength band is, for example, equal to or larger than 400 nanometers (nm) and equal to or smaller than 430 nm (a central wavelength is 415 nm). The thermally denatured region is a region in which heat treatment is performed by an energy device, such as an electrosurgical knife, and a living tissue is thermally denatured. The excitation light that is emitted by the second light source unit 32 is blocked by the cut filter 203 and fluorescence (wavelength of 540 nm) that is generated from the AGEs transmits the cut filter 203, and therefore, it is possible to capture a fluorescence image. The second light source unit 32 is implemented by a collimator lens, a semiconductor laser, such as a purple Laser Diode (LD), a driving driver, and the like.

The light source controller 33 is configured with a processor that is a processing apparatus that includes hardware, such as an FPGA or a CPU, and a memory that is a temporary storage area that is used by the processor. The light source controller 33 controls a light emission timing, light emission intensity, a light emission duration, or the like of each of the first light source unit 31 and the second light source unit 32 based on control data that is input from a control unit 405.

### Configuration of Control Apparatus

A configuration of the control apparatus 4 will be described below.

The control apparatus 4 includes a serial-to-parallel (S/P) converter 401, an image processing unit 402, an input unit 403, a recording unit 404, and the control unit 405.

The S/P converter 401 performs serial-to-parallel conversion on the imaging signal that is received from the endoscope 2 via the first transmission cable 232, and outputs the imaging signal to the image processing unit 402, under the control of the control unit 405. Meanwhile, when the endoscope 2 outputs the imaging signal by an optical signal, it may be possible to arrange, instead of the S/P converter 401, an optical-to-electrical (O/E) converter that converts an optical signal to an electrical signal. Further, when the endoscope 2 transmits the imaging signal by radio communication, it may be possible to arrange, instead of the S/P converter 401, a communication module that is able to receive a radio signal.

The image processing unit 402 is implemented by a processor that includes hardware, such as a CPU, a Graphics Processing Unit (GPU), or an FPGA, and a memory that is a temporary storage area that is used by the processor. The image processing unit 402 performs predetermined image processing on the imaging signal that is input from the S/P converter 401, and outputs the imaging signal to the display apparatus 5, under the control of the control unit 405. The image processing unit 402 generates a white light image from the first imaging signal, and generates a fluorescence image from the second imaging signal. The image processing unit 402 includes an image generation unit 402a, an acquisition unit 402b, an identification unit 402c, an estimation unit 402d, an adjustment unit 402e, and an output unit 402f.

The image generation unit 402a generates a white light image from the first imaging signal that is obtained by applying white light from the first light source unit 31 to a living tissue and imaging return light. Further, the image generation unit 402a generates a fluorescence image from the second imaging signal that is obtained by applying excitation light from the second light source unit 32 to the living tissue and imaging fluorescence. Furthermore, the image generation unit 402a may generate a reference light image from a third imaging signal that is obtained by applying reference light from the first light source unit 31 to the living tissue and imaging return light.

The acquisition unit 402b acquires the white light image, the fluorescence image, and the reference light image from the image generation unit 402a.

The identification unit 402c identifies a position of a marking that is formed by cauterizing the living tissue from positional information on a pixel for which a luminance value is equal to or larger than a first threshold in the fluorescence image. The first threshold is set to a certain value by which fluorescence that is emitted from the AGEs that are generated by thermal denaturation of the living tissue is extractable. As a result, it is possible to identify the position of the marking including the AGEs. Furthermore, the identification unit 402c may identify an incised line that is a line obtained by incising the living tissue, from positional information on a pixel for which a luminance value is equal to or larger than a second threshold in the fluorescence image. Meanwhile, the second threshold is set to a certain value that is larger than the first threshold. As for output of the energy device, output for incision is larger than output for marking, and therefore, a large amount of AGEs are generated at the time of incision and an amount of fluorescence is increased. Therefore, by setting the second threshold to a larger value than the first threshold, it is possible to identify the position of the marking and the incised line in a distinguishable manner.

The estimation unit 402d estimates an incision line that is an auxiliary line for incising the living tissue based on the position of the marking. Meanwhile, when a distal end of the endoscope is located close to the living tissue and only a part of the marking appears in the endoscopic image, the estimation unit 402d may estimate the incision line by comparing positional information on a feature point (a feature point in the image, such as an end portion of a lesion or a bleeding point) of a part that appears in the endoscopic image of the living tissue and positional information on a pixel for which a luminance value is equal to or larger than the first threshold in the fluorescence image.

The adjustment unit 402e performs positional alignment between the white light image and the fluorescence image. The adjustment unit 402e extracts a feature point in the white light image and a feature point in the fluorescence image, and performs positional alignment to align the positions of the feature points. Further, the adjustment unit 402e may perform positional alignment between the white light image and the reference light image. By simultaneously capturing the reference light image and the fluorescence image, it is possible to perform positional alignment between the white light image and the fluorescence image via the reference light image, so that it is possible to improve accuracy of the positional alignment.

The output unit 402f outputs information in which the incision line is superimposed on the white light image. The output unit 402f outputs, for example, a display control signal for causing the display apparatus 5 to display an image in which the incision line is superimposed on the white light image. Further, the output unit 402f may output information in which the incised line is superimposed on the white light image. For example, the output unit 402f outputs a display control signal for causing the display apparatus 5 to display an image in which the incised line is superimposed on the white light image. Meanwhile, the output unit 402f may output information in which the incision line and the incised line are superimposed, in different modes, on the white light image.

The input unit 403 receives input of various kinds of operation on the endoscope system 1, and outputs the received operation to the control unit 405. The input unit 403 is configured with a mouse, a foot switch, a keyboard, a button, a switch, a touch panel, or the like.

The recording unit 404 is implemented by a volatile memory, a non-volatile memory, a Solid State Drive (SSD), a Hard Disk Drive (HDD), or a recording medium, such as a memory card. The recording unit 404 records therein data that includes various kinds of parameters that are needed for operation of the endoscope system 1. The recording unit 404 records therein, for example, positional information on features points in the white light image, the fluorescence image, and the reference light image. Further, the recording unit 404 records therein, for example, positional information on the incision line and positional information on the incised line in a distinguishable manner. Furthermore, the recording unit 404 includes a program recording unit 404a that records therein various kinds of programs for operating the endoscope system 1.

The control unit 405 is implemented by a processor that includes hardware, such as an FPGA or a CPU, and a memory that is a temporary storage area that is used by the processor. The control unit 405 comprehensively controls each of the units that are included in the endoscope system 1.

### Process of Control Apparatus

A process that is performed by the control apparatus 4 will be described below.

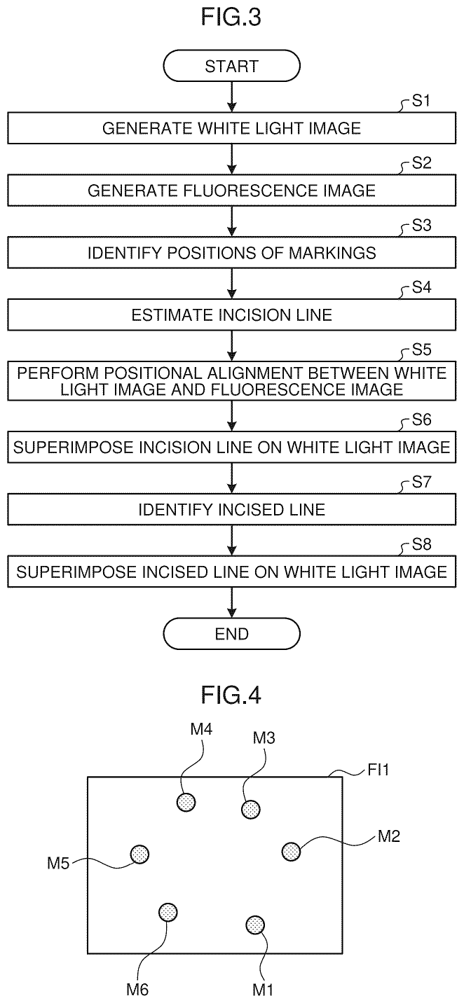

FIG. 3 is a flowchart illustrating an outline of the process that is performed by the control apparatus. As illustrated in FIG. 3, firstly, the image generation unit 402a generates a white light observation image from the first imaging signal that is obtained by applying white light from the first light source unit 31 to a living tissue and performing imaging (Step S1).

Further, the image generation unit 402a generates a fluorescence image from the second imaging signal that is obtained by applying excitation light from the second light source unit 32 to the living tissue and performing imaging (Step S2). FIG. 4 is a diagram illustrating an example of the fluorescence image. As illustrated in FIG. 4, a fluorescence image FIL includes markings M1 to M6.

Subsequently, the identification unit 402c identifies positions of the markings M1 to M6 that are formed by cauterizing the living tissue, from positional information on pixels for which luminance values are equal to or larger than the first threshold in the fluorescence image FI1 (Step S3). The markings M1 to M6 that are formed by cauterizing the living tissue by an energy device contain AGEs that generate fluorescence, and therefore can be identified as positions at which the luminance values are equal to or larger than the first threshold in the fluorescence image FI1.

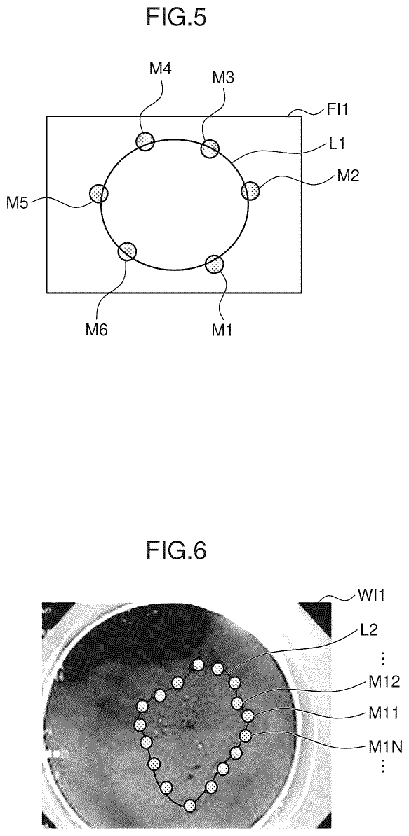

Thereafter, the estimation unit 402d estimates an incision line that is an auxiliary line for incising the living tissue based on the positions of the markings M1 to M6 (Step S4). FIG. 5 is a diagram illustrating an example of the incision line. As illustrated in FIG. 5, the estimation unit 402d estimates a curve that connects the markings M1 to M6 as an incision line L1, for example.

Subsequently, the adjustment unit 402e performs positional alignment between the white light image and the fluorescence image (Step S5).

Further, the output unit 402f outputs a display control signal for causing the display apparatus 5 to display an image in which the incision line is superimposed on the white light image (Step S6). FIG. 6 is a diagram illustrating a state in which the incision line is superimposed on the white light image. As illustrated in FIG. 6, the output unit 402f outputs a display control signal for causing the display apparatus 5 to display an image in which positions of markings M11 to MIN and an incision line L2 are superimposed on a white light image WL1.

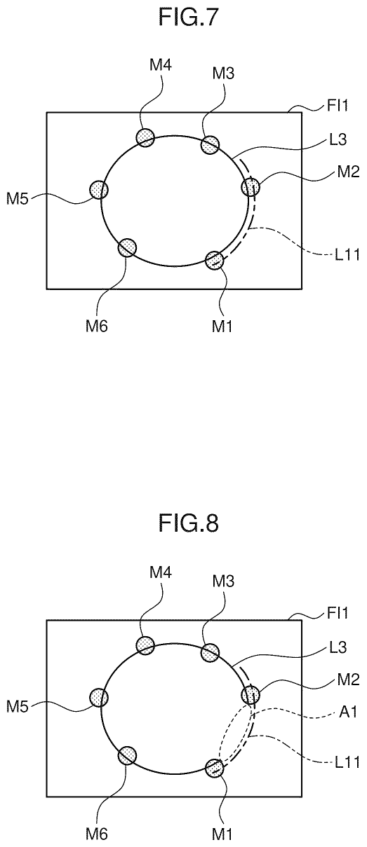

Thereafter, an operator makes an incision around a lesion site by using the incision line as a guideline. In this case, the identification unit 402c identifies an incised line that is a line along which the living tissue is incised from positional information on a pixel for which a luminance value is equal to or larger than the second threshold in the fluorescence image (Step S7). A region in which the living tissue is incised by the energy device contains a large amount of AGEs as compared to the positions of the markings, and therefore can be identified as a position at which a luminance value is equal to or larger than the second threshold that is larger than the first threshold in the fluorescence image FI1. FIG. 7 is a diagram illustrating an example of the incised line. As illustrated in FIG. 7, the identification unit 402c identifies an incised line L11 in the fluorescence image FI1.

Further, the output unit 402f outputs a display control signal for causing the display apparatus 5 to display an image in which the incised line is superimposed on the white light image (Step S8).

Meanwhile, the output unit 402f may output a display control signal for causing the display apparatus 5 to display an image in which the incision line is superimposed on the white light image such that a part of the incision line is deleted in accordance with the incised line. FIG. 8 is a diagram illustrating a state in which a part of the incision line is deleted. As illustrated in FIG. 8, the output unit 402f may delete the incision line in an area A1 in which the incision is already performed in the fluorescence image FI1. Specifically, the output unit 402f may output a display control signal for causing the display apparatus 5 to display an image in which an incision line L3 in which an incision line between the marking M1 and the marking M2 through which the incised line L11 has passed and an incised line L11 are superimposed on the white light image. As a result, it is possible to prevent degradation of visibility due to overlapping of the incision line and the incised line.

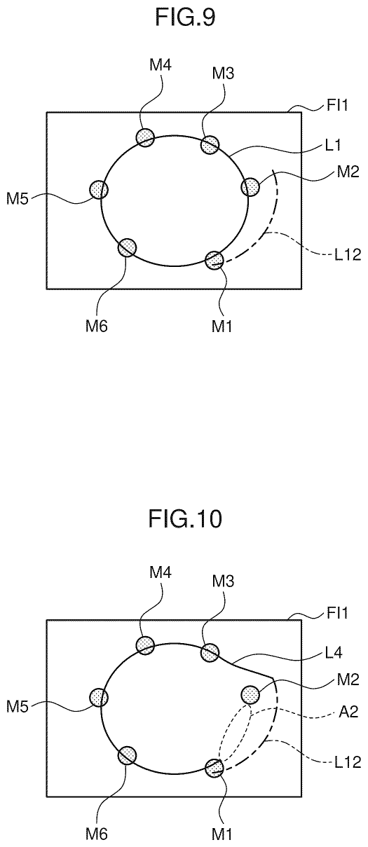

FIG. 9 is a diagram illustrating an example of the incised line. As illustrated in FIG. 9, in some cases, an incised line L12 passes through the marking M1, but thereafter does not pass through the adjacent marking M2. FIG. 10 is a diagram illustrating a state in which a part of the incision line is deleted. As illustrated in FIG. 10, the output unit 402f may delete an incision line in an area A2 in which the incision is already performed in the fluorescence image FI1. Specifically, when the incised line L12 has passed through, for example, a predetermined line (for example, a straight line that connects the marking M2 and a midpoint of a straight line connecting the marking M1 and the marking M3), the output unit 402f may delete the incision line in the area A2. In this case, the output unit 402f may connect an end portion of the incised line L12 and an end portion of an incision line L4. Further, the output unit 402f may output a display control signal for causing the display apparatus 5 to display an image in which the incision line L4 and the incised line L12 are superimposed on the white light image. As a result, it is possible to prevent degradation of visibility due to overlapping of the incision line and the incised line.

According to one embodiment as described above, the incision line is superimposed on the white light image, so that the operator is able to easily recognize the incision line.

Furthermore, because the incised line is superimposed on the white light image, the operator is able to easily recognize the positional relationship between the incised line and the incision line.

### Modification

In the control apparatus 4 according to one modification, the acquisition unit 402b acquires output information of an energy device that is used for marking. The output information may include a type of energy that is output by the energy device. Meanwhile, examples of the type of the energy include a high frequency, an ultrasound wave, and a microwave.

The identification unit 402c sets the first threshold in accordance with the output information that is acquired by the acquisition unit 402b. Further, the identification unit 402c identifies a position of a marking that is formed by cauterizing a living tissue from positional information on a pixel for which a luminance value is equal to or larger than the set first threshold in a fluorescence image.

According to one modification as described above, the first threshold is set in accordance with the output information of the energy device that is used for the marking, so that it is possible to identify the incision line with increased accuracy.

Furthermore, the acquisition unit 402b may acquire output information of an energy device that is used for the incision. In this case, the identification unit 402c sets the second threshold in accordance with the output information that is acquired by the acquisition unit 402b. Moreover, the identification unit 402c identifies an incised line that is a line obtained by incising a living tissue, from positional information on a pixel for which a luminance value is equal to or larger than the second threshold in the fluorescence image. As a result, it is possible to identify the incision line and the incised line in a distinguishable manner in accordance with the output information of the energy device.

Moreover, the control unit 405 may have a function as a learning unit of a learning apparatus that is the control apparatus 4. The control unit 405 may generate a trained model by performing machine learning by using teacher data in which a white light image that is obtained by applying white light to a living tissue and imaging return light and a fluorescence image that is obtained by applying excitation light to the living tissue and imaging fluorescence are adopted as input data and information in which an incision line that is an auxiliary line for incising the living tissue is superimposed on the white light image is adopted as output data. Here, the trained model is formed of a neural network in which each of layers includes one or a plurality of nodes. Furthermore, a type of the machine learning is not specifically limited; however, for example, it is sufficient to prepare teacher data and training data in which a plurality of white light images and fluorescence images of a subject are associated with an image in which an incision line that is estimated from the plurality of white light images and the plurality of fluorescence images is superimposed on a white light image, and perform learning by inputting the teacher data and the training data in a calculation model that is based on a multi-layer neural network. Moreover, as a method of the machine learning, for example, a method based on a Deep Neural Network (DNN) as a multi-layer neural network, such as a Convolutional Neural Network (CNN) or a 3D-CNN, may be used. Furthermore, as the method of the machine learning, a method based on a Recurrent Neural Network (RNN), a Long Short-Term Memory units (LSTM) that is an expanded RNN, or the like may be used. Meanwhile, a learning unit of a different learning apparatus from the control apparatus 4 may implement the above-described functions.

According to the disclosure, it is possible to implement an image processing apparatus, a medical system, an operation method of the image processing apparatus, and a learning apparatus capable of easily recognizing an incision line.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

### Example

Example 1. An image processing apparatus comprising:
a processor comprising hardware, the processor being configured to
   acquire a white light image by applying white light to a living tissue and imaging return light and a fluorescence image by applying excitation light to the living tissue and imaging fluorescence,
   acquire output information of an energy device,
   set a first threshold in accordance with the output information,
   estimate an incision line that is an auxiliary line for incising the living tissue based on positional information on a pixel for which a luminance value is equal to or larger than the first threshold in the fluorescence image,
   perform positional alignment between the white light image and the fluorescence image, and
   output information in which the incision line is superimposed on the white light image.
      Example 2. The image processing apparatus according to Example 1, wherein the processor is further configured to
identify a position of a marking that is formed by cauterizing the living tissue, from the positional information on the pixel for which the luminance value is equal to or larger than the first threshold in the fluorescence image, and
estimate the incision line based on the position of the marking.
   Example 3. The image processing apparatus according to Example 2, wherein the processor is further configured to identify an incised line that is a line obtained by incising the living tissue, from positional information on a pixel for which a luminance value is equal to or larger than a second threshold in the fluorescence image.
   Example 4. The image processing apparatus according to Example 3, wherein the second threshold is larger than the first threshold.
   Example 5. The image processing apparatus according to Example 1, wherein the output information includes a type of energy that is output by the energy device.
   Example 6. The image processing apparatus according to Example 1, wherein the processor is further configured to
set a second threshold in accordance with the output information, and
identify an incised line that is a line obtained by incising the living tissue, form positional information on a pixel for which a luminance value is equal to or larger than the second threshold in the fluorescence image.
   Example 7. The image processing apparatus according to Example 3, wherein the processor is further configured to output information in which the incision line and the incised line are superimposed, in different modes, on the white light image.
   Example 8. The image processing apparatus according to Example 3, wherein the processor is further configured to store therein positional information on the incision line and positional information on the incised line in a distinguishable manner.
   Example 9. The image processing apparatus according to Example 1, wherein the processor is further configured to generate the fluorescence image from an imaging signal that is obtained by applying the excitation light to the living tissue and imaging the fluorescence.
   Example 10. The image processing apparatus according to Example 1, wherein the processor is further configured to
acquire a reference light image by applying reference light with a wavelength that is included in a wavelength band of the white light and that does not include a wavelength band of the fluorescence to the living tissue and imaging return light, and
perform positional alignment between the white light image and the reference light image.
   Example 11. The image processing apparatus according to Example 10, wherein the reference light image and the fluorescence image are simultaneously captured.
   Example 12. The image processing apparatus according to Example 1, wherein the processor is further configured to extract a feature point from the white light image.
   Example 13. The image processing apparatus according to Example 12, wherein the processor is further configured to store therein positional information on the feature point.
   Example 14. The image processing apparatus according to Example 13, wherein the processor is further configured to estimate the incision line by comparing the positional information on the feature point and the positional information on the pixel for which the luminance value is equal to or larger than the first threshold in the fluorescence image.
   Example 15. The image processing apparatus according to Example 1, wherein the first threshold is set to a value by which the fluorescence that is emitted from advanced glycation end products generated by thermal denaturation of the living tissue is extractable.
   Example 16. A medical system comprising:
a light source configured to apply while light and excitation light to a living tissue;
an endoscope that includes an image sensor configured to output a first imaging signal that is obtained by imaging return light of the white light and a second imaging signal that is obtained by imaging fluorescence caused by the excitation light; and
an image processing apparatus that includes a processor configured to generate a white light image from the first imaging signal and a fluorescence image from the second imaging signal, wherein
the processor is further configured to
   acquire output information of an energy device,
   set a first threshold in accordance with the output information,
   estimate an incision line that is an auxiliary line for incising the living tissue based on positional information on a pixel for which a luminance value is equal to or larger than the first threshold in the fluorescence image,
   perform positional alignment between the white light image and the fluorescence image, and
   output information in which the incision line is superimposed on the white light image.
      Example 17. An operation method of an image processing apparatus comprising a processor comprising hardware, the processor being configured to
acquire a white light image by applying white light to a living tissue and imaging return light and a fluorescence image by applying excitation light to the living tissue and imaging fluorescence,
acquire output information of an energy device,
set a first threshold in accordance with the output information,
estimate an incision line that is an auxiliary line for incising the living tissue based on positional information on a pixel for which a luminance value is equal to or larger than the first threshold in the fluorescence image,
perform positional alignment between the white light image and the fluorescence image, and
output information in which the incision line is superimposed on the white light image.
   Example 18. A learning apparatus comprising:
a learning processor configured to generate a trained model by performing machine learning by using teacher data in which a white light image that is obtained by applying white light to a living tissue and imaging return light and a fluorescence image that is obtained by applying excitation light to the living tissue and imaging fluorescence are adopted as input data and information in which an incision line that is an auxiliary line for incising the living tissue is superimposed on the white light image is adopted as output data.
   Example 19. An image processing apparatus comprising:
a processor comprising hardware, the processor being configured to
   acquire a white light image and a fluorescence image for a living tissue,
   acquire output information of an energy device,
   set a first threshold in accordance with the output information,
   estimate an auxiliary line for incising the living tissue based on positional information on a pixel for which a luminance value is equal to or larger than the first threshold in the fluorescence image, and
   generate information in which the auxiliary line is superimposed on the white light image.
      Example 20. The image processing apparatus according to Example 19, wherein the processor is further configured to
identify a position of a marking that is formed by cauterizing the living tissue, from the positional information on the pixel for which the luminance value is equal to or larger than the first threshold in the fluorescence image, and
estimate the auxiliary line based on the position of the marking.
   Example 21. The image processing apparatus according to Example 20, wherein the processor is further configured to identify an incised line obtained by incising the living tissue, from positional information on a pixel for which a luminance value is equal to or larger than a second threshold in the fluorescence image.
   Example 22. The image processing apparatus according to Example 21, wherein the second threshold is larger than the first threshold.
   Example 23. The image processing apparatus according to Example 19, wherein the output information includes a type of energy that is output by the energy device.
   Example 24. The image processing apparatus according to Example 19, wherein the processor is further configured to
set a second threshold in accordance with the output information, and
identify an incised line obtained by incising the living tissue, form positional information on a pixel for which a luminance value is equal to or larger than the second threshold in the fluorescence image.
   Example 25. The image processing apparatus according to Example 21, wherein the processor is further configured to output information in which the auxiliary line and the incised line are superimposed, in different modes, on the white light image.
   Example 26. The image processing apparatus according to Example 21, wherein the processor is further configured to store therein positional information on the auxiliary line and positional information on the incised line in a distinguishable manner.
   Example 27. The image processing apparatus according to Example 19, wherein the processor is further configured to generate the fluorescence image from an imaging signal that is obtained by applying excitation light to the living tissue and imaging fluorescence.
   Example 28. The image processing apparatus according to Example 19, wherein the processor is further configured to
acquire a reference light image by applying reference light with a wavelength that is included in a wavelength band of white light and that does not include a wavelength band of fluorescence to the living tissue and imaging return light, and
perform positional alignment between the white light image and the reference light image.
   Example 29. The image processing apparatus according to Example 28, wherein the reference light image and the fluorescence image are simultaneously captured.
   Example 30. The image processing apparatus according to Example 19, wherein the processor is further configured to extract a feature point from the white light image.
   Example 31. The image processing apparatus according to Example 30, wherein the processor is further configured to store therein positional information on the feature point.
   Example 32. The image processing apparatus according to Example 31, wherein the processor is further configured to estimate the auxiliary line by comparing the positional information on the feature point and the positional information on the pixel for which the luminance value is equal to or larger than the first threshold in the fluorescence image.
   Example 33. The image processing apparatus according to Example 19, wherein the first threshold is set to a value by which the fluorescence that is emitted from advanced glycation end products generated by thermal denaturation of the living tissue is extractable.
   Example 34. The image processing apparatus according to Example 19, wherein the processor is further configured to
acquire the white light image by applying white light to the living tissue and imaging return light, and
acquire the fluorescence image by applying excitation light to the living tissue and imaging fluorescence.
   Example 35. The image processing apparatus according to Example 19, wherein the processor is further configured to
extract a first feature point in the white light image and a second feature point in the fluorescence image, and
align a position of the first feature point with a position of the second feature point to perform positional alignment between the white light image and the fluorescence image.
   Example 36. The image processing apparatus according to Example 20, wherein the first threshold is set in accordance with the output information of the energy device used for the marking.
   Example 37. The image processing apparatus according to Example 21, wherein the second threshold is set in accordance with the output information of the energy device used for incising the living tissue.
   Example 38. A medical system comprising:
a light source configured to apply while light and excitation light to a living tissue;
an endoscope that includes an image sensor configured to output a first imaging signal that is obtained by imaging return light of the white light and a second imaging signal that is obtained by imaging fluorescence caused by the excitation light; and
an image processing apparatus that includes a processor configured to generate a white light image from the first imaging signal and a fluorescence image from the second imaging signal, wherein
the processor is further configured to
   acquire output information of an energy device,
   set a first threshold in accordance with the output information,
   estimate an auxiliary line for incising the living tissue based on positional information on a pixel for which a luminance value is equal to or larger than the first threshold in the fluorescence image,
   generate information in which the auxiliary line is superimposed on the white light image.
      Example 39. An operation method of an image processing apparatus comprising a processor comprising hardware, the processor being configured to
acquire a white light image and a fluorescence image for a living tissue,
acquire output information of an energy device,
set a first threshold in accordance with the output information,
estimate an auxiliary line for incising the living tissue based on positional information on a pixel for which a luminance value is equal to or larger than the first threshold in the fluorescence image, and
generate information in which the auxiliary line is superimposed on the white light image.
   Example 40. A learning apparatus comprising:
a learning processor configured to generate a trained model by performing machine learning by using teacher data in which a white light image and a fluorescence image for a living tissue are adopted as input data and information in which an auxiliary line for incising the living tissue is superimposed on the white light image is adopted as output data.

What is claimed is:

1. An image processing apparatus comprising:
a processor comprising hardware, the processor being configured to
acquire a white light image and a fluorescence image for a living tissue,
acquire output information of an energy device,
set a first threshold in accordance with the output information,
estimate an auxiliary line for incising the living tissue based on first positional information corresponding to a pixel in the fluorescence image having a luminance value equal to or larger than the first threshold, and
generate information in which the auxiliary line is superimposed on the white light image.

2. The image processing apparatus according to claim 1, wherein the processor is further configured to
identify a position of a marking that is formed by cauterizing the living tissue, from the first positional information, and
estimate the auxiliary line based on the position of the marking.

3. The image processing apparatus according to claim 2, wherein the processor is further configured to identify an incised line obtained by incising the living tissue, from second positional information corresponding to a pixel in the fluorescence image having a luminance value equal to or larger than a second threshold.

4. The image processing apparatus according to claim 3, wherein the second threshold is larger than the first threshold.

5. The image processing apparatus according to claim 1, wherein the output information includes a type of energy that is output by the energy device.

6. The image processing apparatus according to claim 1, wherein the processor is further configured to
set a second threshold in accordance with the output information, and
identify an incised line obtained by incising the living tissue, form second positional information corresponding to a pixel in the fluorescence image having a luminance value equal to or larger than the second threshold.

7. The image processing apparatus according to claim 3, wherein the processor is configured to generate information in which the auxiliary line and the incised line are superimposed, in different modes, on the white light image.

8. The image processing apparatus according to claim 3, wherein the processor is further configured to store therein third positional information on the auxiliary line and fourth positional information on the incised line in a distinguishable manner.

9. The image processing apparatus according to claim 1, wherein the processor is further configured to
acquire a reference light image by applying reference light with a wavelength that is included in a wavelength band of white light and that does not include a wavelength band of fluorescence to the living tissue and imaging return light, and
perform positional alignment between the white light image and the reference light image.

10. The image processing apparatus according to claim 9, wherein the reference light image and the fluorescence image are simultaneously captured.

11. The image processing apparatus according to claim 1, wherein the processor is further configured to extract a feature point from the white light image.

12. The image processing apparatus according to claim 11, wherein the processor is further configured to estimate the auxiliary line by comparing a second positional information on the feature point and the first positional information.

13. The image processing apparatus according to claim 1, wherein the first threshold is set to a value by which the fluorescence that is emitted from advanced glycation end products generated by thermal denaturation of the living tissue is extractable.

14. The image processing apparatus according to claim 1, wherein the processor is further configured to
acquire the white light image by applying white light to the living tissue and imaging return light, and
acquire the fluorescence image by applying excitation light to the living tissue and imaging fluorescence.

15. The image processing apparatus according to claim 1, wherein the processor is further configured to
extract a first feature point in the white light image and a second feature point in the fluorescence image, and
align a position of the first feature point with a position of the second feature point to align a position of the white light image with the fluorescence image.

16. The image processing apparatus according to claim 2, wherein the first threshold is set in accordance with the output information of the energy device used for the marking.

17. The image processing apparatus according to claim 3, wherein the second threshold is set in accordance with the output information of the energy device used for incising the living tissue.

18. A medical system comprising:
a light source configured to apply while light and excitation light to a living tissue;
an endoscope that includes an image sensor configured to output a first imaging signal that is obtained by imaging return light of the white light and a second imaging signal that is obtained by imaging fluorescence caused by the excitation light; and
the image processing apparatus according to claim 1, wherein the processor is further configured to generate a white light image from the first imaging signal and a fluorescence image from the second imaging signal.

19. An operation method of an image processing apparatus, the method comprising: acquiring a white light image and a fluorescence image for a living tissue,
acquiring output information of an energy device,
setting a first threshold in accordance with the output information,
estimating an auxiliary line for incising the living tissue based on positional information corresponding to a pixel in the fluorescence image having a luminance value equal to or larger than the first threshold, and
generating information in which the auxiliary line is superimposed on the white light image.

20. A learning apparatus comprising:
a learning processor configured to generate a trained model by performing machine learning by using teacher data in which a white light image and a fluorescence image for a living tissue are adopted as input data and information in which an auxiliary line for incising the living tissue is superimposed on the white light image is adopted as output data.
